(19) United States
(12) Patent Application Publication
(10) Pub. No.: US 2024/0018451 A1
(43) Pub. Date: Jan. 18, 2024

(54) DEVELOPING A HYBRID MODEL OF A BIOCHEMICAL FERMENTATION PROCESS

(71) Applicants: The Texas A&M University System, College Station, TX (US); Kaneka Americas Holding, Inc., Pasadena, TX (US)

(72) Inventors: Costas Kravaris, College Station, TX (US); Joseph Kwon, College Station, TX (US); Parth Shah, Bryan, TX (US); Mohammed Ziyan Sheriff, College Station, TX (US); Mohammed Saad Faizan Bangi, College Station, TX (US); Chiranjivi Botre, Pasadena, TX (US); Junichi Hirota, Pasadena, TX (US)

(73) Assignees: The Texas A&M University System, College Station, TX (US); Kaneka Americas Holding, Inc., Pasadena, TX (US)

(21) Appl. No.: 18/037,296

(22) PCT Filed: Nov. 16, 2021
(86) PCT No.: PCT/US2021/072425

§ 371 Date: May 16, 2023

Related U.S. Application Data
(60) Provisional application No. 63/114,297, filed on Nov. 16, 2020.

(51) Int. Cl. C12M 1/00; G06N 3/04

(52) U.S. Cl. CPC C12M 21/00; G06N 3/04

(57) ABSTRACT

A system for producing a product is disclosed. The system includes a production facility for producing a product using a chemical process involving chemical reactions, and an information processing device comprising a computer processor that simulates, using a hybrid model, the chemical reactions in the chemical process that produces the product to obtain a predicted output, wherein the hybrid model is a combination of a first-principles model and a data-driven model, determines, using an observer model, expected concentrations and levels of all substrates for the simulated process, sets derived optimal conditions for the chemical process based on the estimated concentrations and levels of all substrates, and predicts future production results based on a current status of the production facility.

### BACKGROUND

In the fields of chemical and bioprocess engineering, reactors may be used to control a chemical reaction. Chemical reactions include, but are not limited to, synthesis reactions, decomposition reactions, biological reactions, and biochemical reactions. The chemical reaction takes place inside the reactor. A reactor system may include a reactor, a control system, and sensors that monitor reaction parameters. The control system receives data from the sensors, compares the data with desired target values, and derives command functions that are used to control the reaction by operation of control components, such as valves and switches.

In a bioreactor, microorganisms or biochemicals consume raw materials while performing a desired biological function. This type of biological process may convert the raw materials to a finished product that is passed from the bioreactor. In some biological processes, microorganisms consume raw materials resulting in cell growth. As the cells grow, the population of the microorganisms may multiply. Cell multiplication may result in the production of biomass. In some other biological processes, biochemicals such as enzymes may bind raw materials like substrates or intermediates to initiate a chemical reaction. The enzymatic reaction may produce a product, and the product is released from the enzyme. In some instances, biomass production and enzymatic reactions may be included together as a desired biological function, to produce finished goods from a bioreactor.

### SUMMARY

This Summary is provided to introduce a selection of concepts that are further described in the Detailed Description. This Summary is not intended to identify key or essential features of the claimed subject matter, nor is it intended to be used as an aid in limiting the scope of the claimed subject matter.

In one aspect, embodiments disclosed relate to a system for producing a product, the system comprising a production facility for producing a product using a chemical process involving chemical reactions, and an information processing device comprising a computer processor that simulates, using a hybrid model, the chemical reactions in the chemical process that produces the product to obtain a predicted output, wherein the hybrid model is a combination of a first-principles model and a data-driven model; determines, using an observer model, expected concentrations and levels of all substrates for the simulated process; sets derived optimal conditions for the chemical process based on the estimated concentrations and levels of all substrates; and predicts future production results based on a current status of the production facility.

In one aspect, embodiments disclosed herein relate to a computer-implemented method for predicting substrate concentrations in a chemical process to produce a chemical product, comprising simulating, using a hybrid model, chemical reactions in the chemical process to obtain a predicted output, wherein the hybrid model is a combination of a first-principles model and a data-driven model; determining, using an estimation model, an estimation of all concentrations and levels for the simulated chemical process; setting derived optimal conditions for the chemical process based on the estimated substrate concentrations and predicting future production results of the product based on a current status of the production facility.

Other aspects and advantages of the claimed subject matter will be apparent from the following Detailed Description and the appended Claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIG. 1 shows a system, including a reactor and a processing device comprising a computer processor, according to one or more embodiments.

FIG. 2 shows a hybrid model in accordance with one or more embodiments.

FIG. 3 shows a flowchart in accordance with one or more embodiments.

FIG. 4 shows a GUI in accordance with one or more embodiments.

FIG. 5 shows an example flowchart for the hybrid model algorithm in accordance with one or more embodiments.

FIG. 6 shows a computer system in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

One or more embodiments relates to a hybrid model, i.e., a first-principles model combined with one or more data-driven models, for a full-scale bio-fermentation process with a volume of over 100,000 gallons. More specifically, embodiments disclosed herein use an improved first-principles, which is obtained by adding additional components and parameters to its equations. Also, the data-driven models can be Neural networks, Support vector machines, Multivariate adaptive regression splines, Ordinary least squares, Stepwise regression, Latent variable methods, Fuzzy systems, or Genetic algorithms. In one or more embodiments, critical parameters in the improved first-principles model which highly influence its outputs are identified using local and global sensitivity analysis. These time-varying parameters are estimated using a data-clustering approach, and approximated using a deep neural network (DNN) which was then combined with the improved first-principles model to build a hybrid model.

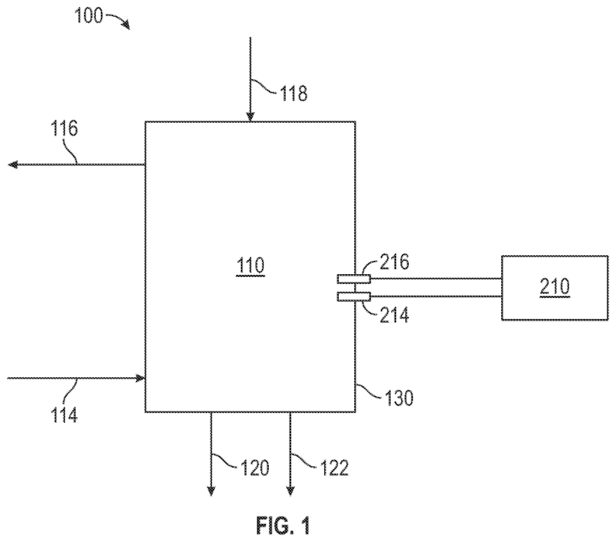

FIG. 1 shows a system 100 according to one or more embodiments. In one or more embodiments, the system 100 of FIG. 1 may be employed in any production facility, such as a chemical plant, to make a chemical product. The system 100 is suitable for a chemical process, such as a chemical process to produce a chemical product. The system 100 includes a reactor 110 and an information processing device 210. System 100 may include one or more feed and product streams. A gas feed stream 114 is coupled to the reactor 110 and comprises a gas, to be described, and is introduced into the reactor. A solid/liquid feed stream 118 is coupled to the reactor and may comprise a solid, a liquid, or a combination thereof, to be described. Solid/liquid feed stream 118 is introduced into the reactor. An off-gas stream 116 may be coupled to the reactor and comprises an off-gas, to be described, and passes from the system 100. An effluent stream 122 is coupled to the reactor and comprises an effluent, to be described, and passes from the system 100. A chemical product stream 120 is coupled to the reactor and comprises a chemical product, to be described, and passes from the system 100. The reactor 110 is defined by a reactor chamber 130. The reactor chamber may include a heat exchanger (not shown), such as a water jacket around reactor chamber 130 configured to receive and pass liquid for heating or cooling.

In one or more embodiments, the system includes one or more selected from the group consisting of a gas feed stream, a solid/liquid feed stream, an off-gas stream, an effluent stream, and a chemical product stream. For example, FIG. 1 shows the aforementioned streams. As another example, the system may include a gas feed stream, a solid/liquid feed stream, an effluent stream, and a chemical product stream. As yet another example, the system may include a gas feed stream, a solid/liquid feed stream, an off-gas stream, and an effluent stream.

FIG. 1 shows a processing device having a computer processor 210 in system 100 and one or more sensors, including, but not limited to one or more probes. The probes may be configured to monitor pH, temperature, fluid level, gas level, pressure, or other reactor parameters. FIG. 1 shows a first probe 214 and a second probe 216 that are coupled to the reactor chamber 130. The sensor may be included inside the reactor chamber, coupled to the reactor chamber (shown in FIG. 1), or outside the reactor chamber, depending on the function of the sensor. Other sensors may include, for example, a flow rate sensor coupled to solid/liquid feed stream (such as 118 in FIG. 1) that is configured to monitor a flow rate. The one or more sensors may be in signal communication with the processing device comprising a computer processor. The signal communication may be a wired communication, a wireless communication, or a combination thereof.

In one or more embodiments, the reactor is a chemical reactor. In one or more embodiments, the reactor is a bioreactor. When the reactor is a bioreactor, it may be a fermenter, such as a batch fermenter, a continuous fermenter, or a recycle reactor. Suitable examples of reactors include, but are not limited to, a tubular reactor, a bubble column reactor, a bubble cap reactor, a plug flow reactor, an airlift reactor, a two-stage airlift reactor, a fluidized bed reactor, a stirred tank reactor, a Heinze-type reactor, a fixed bed reactor, a packed bed reactor, a stainless steel fixed bed reactor (for example, with electric heater), a microwave reactor, and a photoreactor.

In addition to reactor components previously described, the reactor may include an agitator, a sparger, an antifoam stream, an acid stream, a base stream, or a combination thereof.

A chemical process takes place within the reactor. Types of chemical processes include, but are not limited to, a catalytic process, an enzymatic process, a stoichiometric process (such as not catalyzed), a polymerization process, a fermentation process (a “bio-fermentation” process), a biomass production process, and a combination thereof.

The reactor uses materials during a chemical process to produce a chemical product. Thus, the reactor is configured to retain materials for the chemical process. Materials for the chemical process include, but are not limited to, a catalyst, a reactant, an enzyme, an enzyme-substrate, a monomer, an activator, a microorganism, a biomass, a food source, a gas, a solvent, or a combination thereof. The term “food source” relates to a carbon-based material for the growth of microorganisms, for example, starch or sugar such as mannose, sorbitol, glucose, fructose, or lactose. The term enzyme-substrate is used to differentiate from the term substrate. A “substrate” is a material to be used in a reactor. An “enzyme-substrate” is a molecule upon which an enzyme acts. Thus, an enzyme-substrate may be a substrate. Materials for use in chemical processes and reactors are well appreciated in the art.

The gas feed stream comprises a gas. The gas may include, but is not limited to, air, oxygen, nitrogen, argon, helium, carbon monoxide, carbon dioxide, hydrogen, ozone, ethylene, a combination thereof, or other suitable gas. In some instances, the gas is a reactant, such as oxygen, carbon monoxide, hydrogen, ozone, or a hydrocarbon such as ethylene. In some instances, the gas is inert, such as nitrogen, argon, helium, or other suitable noble gas. In some instances, the gas is a growth agent of food source, for example, in a bioreactor, such as oxygen, carbon monoxide, or carbon dioxide. When a microorganism is included in a reactor, the gas used may relate to the aerobic or anaerobic respiration of said microorganism.

The solid/liquid feed may comprise a liquid, such as a solvent. The solvent may include, but is not limited to, acetone, acetic acid, ethyl acetate, pentane, hexane, heptane, dichloromethane, chloroform, methanol, ethanol, isopropanol, tetrahydrofuran, acetonitrile, dimethylformamide, toluene, dimethylsulfoxide, water, or a combination thereof. The solid/liquid feed may comprise a solid, such as one or more materials for the chemical process. In one or more embodiments, the solid/liquid feed comprises a solvent.

The off-gas stream includes an off-gas. The off-gas is a gas that is expelled from the reactor. Thus, it is a type of gas product stream that may be collected, further processed, or re-routed to the reactor. The off-gas may be a gas that is introduced into the reactor and is expelled, or a byproduct or product of a chemical process. For example, a byproduct or a chemical product off-gas may include, but is not limited to, hydrocarbons such as ethylene, carbon dioxide, hydrogen, or oxygen.

The effluent stream includes an effluent. The effluent is a liquid, a solid, or a liquid and a solid that is expelled from the reactor. The effluent may be a liquid or a solid that is introduced into the reactor and is expelled, or a byproduct or product of a chemical process. For example, an effluent may include, but is not limited to, solvent, biomass, or intractable reaction material. Various other examples of effluents from a chemical process would be appreciated by one of ordinary skill in the art.

The chemical product stream includes a chemical product of the chemical process. Various chemical products from a chemical process in a reactor are appreciated in the art.

A chemical product from a reactor may include a polymer, a hydrocarbon, a synthetic natural product, or various other chemical products. As a non-limiting example, a hydrocarbon may include xylene, toluene, benzene, ethylbenzene, polyethylene, polypropylene, polybutene, polyisobutylene, polymethylpentene, ethylene propylene rubber, ethylene propylene diene monomer rubber, and poly alpha-olefins.

When a bioreactor is used, a chemical product may include an organism that is grown, such as yeast, bacteria, or animal or plant cells, or the chemical product may include a pharmaceutical, a vaccine, a vitamin, a coenzyme, or an antibody. As a non-limiting example, a vitamin or a coenzyme may include adenosine triphosphate (ATP), nicotinamide adenine dinucleotide (NAD), nicotinamide adenine dinucleotide phosphate (NADP), flavin adenine dinucleotide (FAD), thiamin (various forms of vitamin B₁), pyridoxal phosphate (vitamin B₆), cobalamin (various forms of vitamin B₁₂), retinol (vitamin A₁), biocytin, coenzyme A, coenzyme b, coenzyme Q10, coenzyme M, tetrahydrofolate, glutathione, heme (various forms), lipoamide, methanofuran, and nucleotide sugars.

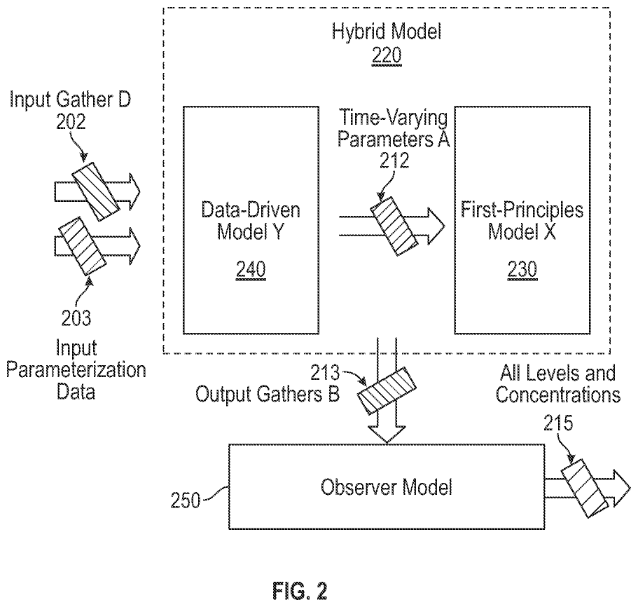

Modelling a chemical process, such as for example a bio-fermentation process, to predict the production of a chemical product for a chemical or plant production facility is a challenging task given the complex interactions that occur within the chemical process. FIG. 2 shows a data-driven and machine learning (ML)-based hybrid model used to simulate the chemical process that results in a chemical product, as described above. More specifically, the model structure used in FIG. 2 simulates the chemical process that occurs in the reactor in order to allow for adjustments to be made to the feed rate of the gas or solid/liquid feeds as described above into the reactor for optimal production at the production facility. FIG. 2 depicts inputs (202, 203) into a hybrid model (220), the output (213) of which is received by an estimate model (250). Each of these components of FIG. 2 is described in detail below.

In one or more embodiments, the hybrid model (220) consists of a first-principles model (230) and a data-driven model (240). The first-principles model (230) is a kinetic model that uses first-principles such as mass and energy conservation laws, kinetic laws, thermodynamic laws, etc., and is able to capture the essential dynamics of the chemical process using these laws of nature. Empirical formulations which represent data that is able to be modelled are introduced in the first-principles model (230). The overall accuracy of the first-principles model is dependent on these empirical relationships. In one or more embodiments, the first-principles model (230) outputs substrate concentrations based on the input parametrization data (203). Input parametrization data (203) are estimates of any of the parameters or variables associated with the gas feed, solid/liquid feed, biomass, additional chemical concentrations, feed flow rates of any of the substrates, growth rate of the substrates, volume, product concentration, etc. Input gather D (202) may be the materials retained by the reactor in order to perform the chemical process as described above, along with online sensor measurements such as temperature and feed flow rates.

In one or more embodiments, the first-principles model (230) and input parametrization data (203) are estimated. Specifically, in order to obtain accurate prediction of output concentrations, accurate parameter estimates of the input parametrization da (203) is vital. The input parametrization data (203) is read and smoothened, growth rates are calculated and the SSE (sum of the square error) is minimized. Using estimated growth rate parameters and ODE parameters guesses, optimized parameters are calculated. The optimized parameters are estimated by solving differential equations using an optimization solver. In one or more embodiments, the hybrid models (220) may employ one or more of the following equations:

[symbol]

where V is volume, Fₖ is the feed flow rate of a substrate into the reactor, P is the product concentration, I is an intermediate concentration, B is the biomass, S₁ and S₂ are the substrate concentrations, Y_{B/S1}, Y_{B/S2}, and Y_{B/I} refer to the yield coefficients of biomass associated with each component, and μᵢ is growth rate of each component. aᵢ is the coefficient linked to the growth rate responsible for increase in product, kᵢ represents the growth rate term associated with I and S₂, while β represents the non-growth term responsible for increase in P. It should be noted that the coefficients linked to the growth rate, kᵢ, incorporate temperature dependence through Arrhenius equation as follows:

$${k}_{i} = {c}_{i} {e}^{- \frac{{E}_{{a}_{i}}}{R \times T}}$$

where cᵢ is the pre-exponential factor, Eₐᵢ is the activation energy, R is the universal gas constant and T refers to the temperature.

In one or more embodiments, the first-principles model (230) employed by embodiments disclosed herein is an improved first-principles model that is augmented by adding additional components and parameters to its equations based on process knowledge acquired from literature studies and experimental evidence. An additional state for X₂ which is expected to play a role similar to the role played by oxygen was incorporated, along with terms in the substrate and product equations to account for the impact of addition of catalyst where X₁ is the catalyst which is known to increase the consumption of substrate and increase production.

$$\frac{{dS}_{2}}{dt} = {k}_{1} {μ}_{{S}_{1}} B - \frac{{μ}_{{S}_{2}} B}{{Y}_{B / {S}_{2}}} - \frac{{F}_{in}}{V} ( {S}_{2} - {S}_{{2}_{initial}} ) - {p}_{1} {X}_{1} \\ \frac{dP}{dt} = ( {α}_{1} {μ}_{{S}_{1}} + {α}_{2} {μ}_{{S}_{2}} + {α}_{3} {μ}_{I} ) B + β B - \frac{{F}_{in}}{V} P + {p}_{2} {X}_{1} \\ \frac{{dX}_{2}}{dt} = {k}_{L} a ( {X}_{{2}_{max}} - {X}_{2} ) - {q}_{{X}_{2}} B$$

where p₁ and p₂ are empirical coefficient that allow X₁ to be incorporated as consumption and production terms in the equations for substrate 2 and product, respectively. X₂ₘₐₓ is the maximum valued of X₂ during a particular batch run, k_{La} and q_{X2} are the mass transfer coefficient, and uptake rate of X₂, respectively.

Critical parameters in the improved first-principles model which highly influence its outputs are identified by performing local and global sensitivity analysis. Parameters like yield coefficients associated with substrate, maximum specific growth rate, and half velocity of the components, are the time-varying parameters identified from the sensitivity analysis. These are estimated using a data-clustering approach, and approximated using the data-driven model to obtain more accurate prediction of behaviour of the chemical process. These obtained parameters are the outputs of the data-driven model (240) and are used in the improved first-principles model (230).

Sensitivity analysis is performed to understand the effect of parameters and initial conditions on output concentrations. Specifically, sensitivity analysis may be performed by calculating a sensitivity matrix based on the differentials of output with respect to the input parametrization data. In one or more embodiments, the sensitivity matrix is as shown below.

$$S = [ \begin{matrix}\partial {y}_{1} ( {t}_{1} ) / \partial {θ}_{1} & … & \partial {y}_{1} ( {t}_{1} ) / \partial {θ}_{{n}_{0}} \\ ⋮ & ⋱ & ⋮ \\ \partial {y}_{1} ( {t}_{{n}_{i}} ) / \partial {θ}_{1} & … & \partial {y}_{1} ( {t}_{{n}_{i}} ) / \partial {θ}_{{n}_{0}} \\ ⋮ & ⋱ & ⋮ \\ \partial {y}_{{n}_{y}} ( {t}_{1} ) / \partial {θ}_{1} & … & \partial {y}_{{n}_{y}} ( {t}_{1} ) / \partial {θ}_{{n}_{0}} \\ … & ⋱ & ⋮ \\ \partial {y}_{{n}_{y}} ( {t}_{{n}_{i}} ) / \partial {θ}_{1} & … & \partial {y}_{{n}_{y}} ( {t}_{{n}_{t}} ) / \partial {θ}_{{n}_{0}}\end{matrix} ]$$

For each output concentration, D-optimality criterion values are calculated, and parameter sets are selected based on descending order of those values. A combined sensitivity analysis is also performed considering all of the output concentrations. In the combined analysis, weights are assigned in multiples for certain outputs over others. For example, substrate S₂ and product P may be given 5 times the weight as compared to other output concentrations.

Based on the results of the sensitivity analysis, the most important input parameterization data are selected, and data clusters are formed. These parameters are estimated inside these clusters to decide which parameter is time-varying, making it a candidate for use inside a neural network for improved prediction of output concentrations. The time-varying parameters (212) are selected based on the clustering results.

As noted above, the hybrid model (220) may employ a data-driven model (240) that relates the inputs with the time-varying parameters used in the first-principles model (230). In one or more embodiments, the data-driven model (240) may be a deep neural network (DNN). With respect to neural networks, for example, a neural network may include one or more hidden layers, where a hidden layer includes one or more neurons. A neuron may be a modelling node or object that is loosely patterned on a neuron of the human brain. In particular, a neuron may combine data inputs with a set of coefficients, i.e., a set of network weights for adjusting the data inputs. These network weights may amplify or reduce the value of a particular data input, thereby assigning an amount of significance to various data inputs for a task being modeled. Through machine learning, a neural network may determine which data inputs should receive greater priority in determining one or more specified outputs of the neural network. Likewise, these weighted data inputs may be summed such that this sum is communicated through a neuron's activation function to other hidden layers within the neural network. As such, the activation function may determine whether and to what extent an output of a neuron progresses to other neurons where the output may be weighted again for use as an input to the next hidden layer.

Turning to recurrent neural networks, a recurrent neural network (RNN) may perform a particular task repeatedly for multiple data elements in an input sequence (e.g., a sequence of well log data), with the output of the recurrent neural network being dependent on past computations. As such, a recurrent neural network may operate with a memory or hidden cell state, which provides information for use by the current cell computation with respect to the current data input. For example, a recurrent neural network may resemble a chain-like structure of RNN cells, where different types of recurrent neural networks may have different types of repeating RNN cells. Likewise, the input sequence may be time-series data, where hidden cell states may have different values at different time steps during a prediction or training operation. For example, where a deep neural network may use different parameters at each hidden layer, a recurrent neural network may have common parameters in an RNN cell, which may be performed across multiple time steps. To train a recurrent neural network, a supervised learning algorithm such as a backpropagation algorithm may also be used. In some embodiments, the backpropagation algorithm is a backpropagation through time (BPTT) algorithm. Likewise, a BITT algorithm may determine gradients to update various hidden layers and neurons within a recurrent neural network in a similar manner as used to train various deep neural networks. In some embodiments, a recurrent neural network is trained using a reinforcement learning algorithm such as a deep reinforcement learning algorithm. For more information on reinforcement learning algorithms, see the discussion below.

Embodiments are contemplated with different types of RNNs. For example, classic RNNs, long short-term memory (LSTM) networks, a gated recurrent unit (GRU), a stacked LSTM that includes multiple hidden LSTM layers (i.e., each LSTM layer includes multiple RNN cells), recurrent neural networks with attention (i.e., the machine-learning model may focus attention on specific elements in an input sequence), bidirectional recurrent neural networks (e.g., a machine-learning model that may be trained in both time directions simultaneously, with separate hidden layers, such as forward layers and backward layers), as well as multidimensional LSTM networks, graph recurrent neural networks, grid recurrent neural networks, etc. With regard to LSTM networks, an LSTM cell may include various output lines that carry vectors of information, e.g., from the output of one LSTM cell to the input of another LSTM cell. Thus, an LSTM cell may include multiple hidden layers as well as various pointwise operation units that perform computations such as vector addition.

Furthermore, the size of the LSTM network may depend on the specific application. For simple geological layers, a limited number of hidden layers may be needed. For complex geological structures, a large number of hidden layers may be used to deal with the varying settings and complexity of the reservoir.

Turning to reinforcement learning, a reinforcement learning algorithm may train a machine-learning model to make a sequence of decisions. For example, a recurrent neural network may be trained by a reinforcement learning algorithm to achieve a predetermined objective in a possibly uncertain, potentially complex environment. In particular, the predetermined objective may be an optimal set of logging parameters for a logging tool in a wellbore, an optimal set of drilling parameters for producing a particular well path, and/or an accurate geological model of one or more subterranean formations. Thus, a reinforcement learning algorithm may employ a trial-and-error procedure to determine a solution to a complex problem. For example, a reinforcement learning algorithm may include a reward function that teaches a recurrent neural network to follow certain rules, while still allowing the machine-learning model to retain information learned from well log data. In training a recurrent neural network, some embodiments include storing multiple recurrent networks, e.g., a pre-trained RNN that learns to predict well log data and a reward RNN that is trained to predict the rules for the pre-trained RNN.

In some embodiments, a deep reinforcement learning algorithm operates in combination with an LSTM network. The deep reinforcement learning algorithm may be used to optimize the position and direction of a well path in order to improve interwell LWD imaging of a formation. The size of the hidden layers updated by a reinforcement learning algorithm may depend on the complexity of the subsurface structure. The more complex the subsurface structure is, the more complex the LSTM network structure may be to predict image data accurately within an interwell region.

In some embodiments, other types of machine learning algorithms (e.g., machine-learning algorithms) may be used to train a model, such as a backpropagation algorithm. In a backpropagation algorithm, gradients are computed for each hidden layer of a neural network in reverse from the layer closest to the output layer proceeding to the layer closest to the input layer. As such, a gradient may be calculated using the transpose of the weights of a respective hidden layer based on an error function (also called a “loss function”). The error function may be based on various criteria, such as mean squared error function, a similarity function, etc., where the error function may be used as a feedback mechanism for tuning weights in the electronic model. One example of a backpropagation algorithm is a Levenberg-Marqardt algorithm. In some embodiments, a machine-learning model is trained using multiple epochs. For example, an epoch may be an iteration of a model through a portion or all of a training dataset. As such, a single machine-learning epoch may correspond to a specific batch of training data, where the training data is divided into multiple batches for multiple epochs. Thus, a machine-learning model may be trained iteratively using epochs until the model achieves a predetermined level of prediction accuracy. Thus, better training of a model may lead to better predictions by a trained model.

Continuing with FIG. 2, in one or more embodiments, the data-driven DNN model is pre-trained with the estimated parameter values that are calculated from the clustering datasets. Once the neural network structure is set up, the output y, error e, and the Jacobian matrix are calculated. The weights and biases of the neural network are iteratively updated using an error function, e.g., a Levenberg-Marqardt algorithm, until tolerance of sum squared error (SSE) is less than a predetermined value. Weights and biases of the DNN are updated by calculating gradient using the Levenberg-Marqardt algorithm as follows:

wₖ₊₁=wₖ−(Jₖ^{T}Jₖ+μ_{Train}I)⁻¹JₖEₖ

bₖ₊₁=bₖ−(Jₖ^{T}Jₖ+μ_{Train}I)⁻¹JₖEₖ

Output y is recalculated, and the process continues until error e<tolerance.

| | |
|---|---|
| Output from First | |
| principles model: X | Actual output: Y |

Error e_{q} = Yₖ − Xₖ
SSE = Σ_{q=1}^{Q}(e_{q}^{T})(e_{q}) where Q is number of measurements.

In one or more embodiments, the output gathers B (213) of the hybrid model (220) along with the input gathers D (202) are fed into an estimation model (250). The output gathers B (213) of the hybrid model represent optimal conditions or condition candidates of the chemical process that result in maximization of the product amount and/or profitability. The observer model (250) is a model that estimates the all concentrations and levels more accurately than the first-principles model (230). In one or more embodiments, the estimation model may employ one or more of the following equations to estimate output concentrations of substrates fed into the reactor of a chemical system.

\hat{x}(t)=Ax(t)+Bu(t)

y(t)=Cx(t),

\hat{x}(t)=(A−KC)\hat{x}(t)+Bu(t)+Ky(t)

ŷ(t)=C\hat{x}(t).

where x, y, u are the state vector, output vector, and the input vector, respectively, of the reactor system. \hat{x}, ŷ are the estimations of the state vector and output vector, respectively, obtained from the estimation model. A, B, C are the state matrix, input matrix, and the output matrix, respectively, in the state-space model of the reactor system. K is the gain matrix of the estimation model.

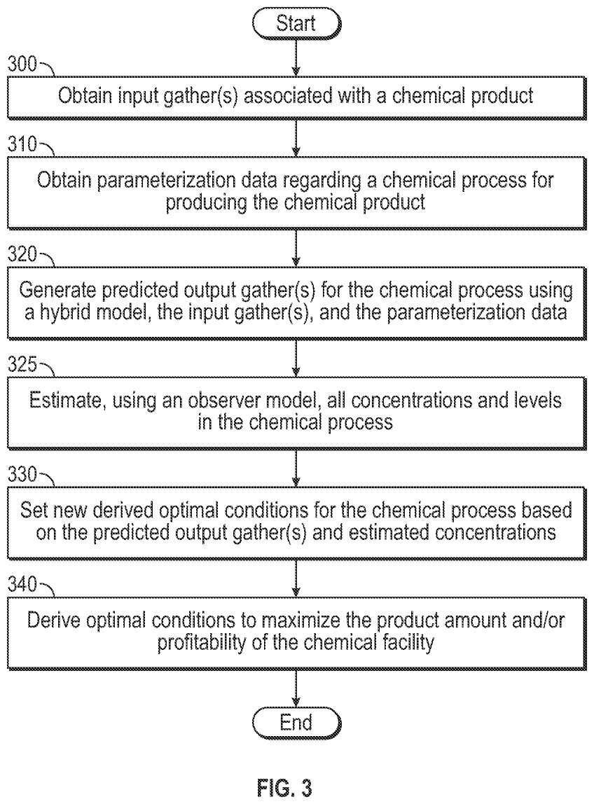

Turning to FIG. 3, FIG. 3 shows a flowchart in accordance with one or more embodiments. Specifically, FIG. 3 describes a general method that uses artificial intelligence to predict output concentrations and levels for simulating a chemical process of a production facility. One or more blocks in FIG. 3 may be performed by one or more components (e.g., information processing device 210, FIG. 1) as described in FIGS. 1 and 2. While the various blocks in FIG. 3 are presented and described sequentially, one of ordinary skill in the art will appreciate that some or all of the blocks may be executed in different orders, may be combined or omitted, and some or all of the blocks may be executed in parallel. Furthermore, the blocks may be performed actively or passively.

Initially, in Block 300, input gather(s) associated with a chemical product to be produced at a production facility are obtained. The input gather(s) may be materials retained by the reactor to produce the chemical product, such as a catalyst, a reactant, an enzyme, an enzyme-substrate, a monomer, an activator, a microorganism, a biomass, a food source, a gas, a solvent, or a combination thereof. Next, in Block 310, parametrization data regarding a chemical process for producing the chemical product is obtained. Parametrization data may include the feed rate of substrates, the growth rate of substrates, reactor volume, yield associated with each substrate, chemical concentrations of added substrates, etc.

Using the input data obtained in Blocks 300 and 310, predicted output gather(s) are generated for the chemical process using a hybrid model that employs a first-principles model and a DNN model to derive optimal conditions to maximize the product amount and/or profitability for optimal product production in Block 320. The hybrid model may be controlled based on the devised optimal conditions. That is, the DNN model inside the hybrid model may be iteratively updated until a tolerance of a SSE is less than a predetermined value.

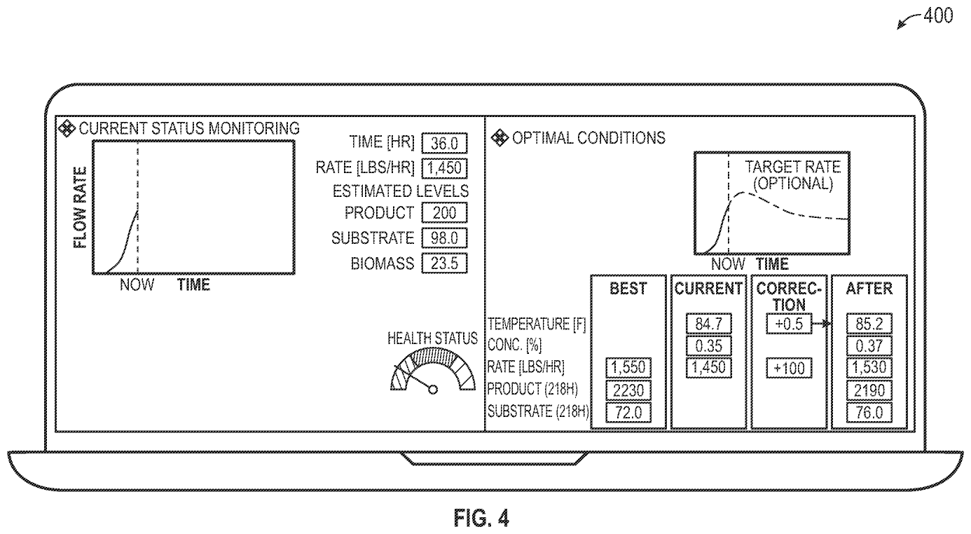

In Block 325, an estimation function or model is used to estimate the important concentrations of the substrates in the chemical process based on the output of the hybrid model, i.e., the optimal conditions for the chemical process obtained from the hybrid model simulation of the chemical process. At this stage, although not shown in FIG. 3, the estimated concentrations and outputs of the hybrid model may be displayed on a GUI dashboard or other display for an operator to observe and act upon. An example of such a GUI dashboard is discussed below and shown in FIG. 4. The estimation function or model employs an open-loop multi-rate observer based on the hybrid model, where states are re-initialized when measurements are available.

In one or more embodiments, the estimation model is further configured to estimate a current status of the production facility. The currents status of the production facility may indicate a health status of the production facility that represents the quality of the current batch process run, i.e., as healthy, intermediate, or needs attention/unhealthy, utilizing information regarding the fermentation status and/or current operating cost.

In Block 330, one or more commands are transmitted to the system (100 in FIG. 1) based on one or more output gathers and/or estimated substrate concentrations in accordance with one or more embodiments. In one or more embodiments, the command sets new derived optimal conditions for the chemical process described with respect to FIG. 1. The command may be sent by an operator that determines how to adjust the feed rate into the reactor (110) based on the data displayed on the GUI, for example. Alternatively, in one or more embodiments, the command may be sent by the information processing device (210) automatically based on the results displayed in the GUI. Based on the output of one or more recurrent neural networks, the command may be fashioned corresponding to a particular parameter value. Thus, the command may be a control signal, e.g., generated by the system (100, 210), or a network message that adjusts the feed rate of substances into the reactor (110).

The simulated chemical process and derived optimal conditions are used to adjust the feed rates of substrate and catalyst, and temperature of reactor which results in the ability to maximize the product amount and/or profitability of production facility making the chemical product (Block 340). An optimization problem can be solved in order to maximize product amount and/or minimize the cost, and maintain the substrate concentration at the desired setpoint, while accounting for all other plant constraints. This results in increase production and lower operating cost of the production facility.

As noted above, between Block 325 and 330 in FIG. 3, the predicted output gather(s) and the estimated substrate concentrations may be displayed for operator observation. FIG. 4 shows an example of a graphical user interface (GUI) (400) that may be used to display the results from the hybrid model simulation of the chemical process. The GUI may be a dashboard that provides an operator with information to check the chemical process health, and set optimal conditions for the feed rate of substrates into the reactor. In one or more embodiments, the GUI includes two regions (areas) of displayed data: a current status monitoring (left side of the GUI) region and a region displaying optimal conditions (right side of the GUI) of the production facility.

The current status monitoring region may display a graph showing the current substrate flow rate into the reactor of the production facility. An icon showing the current health status of the production facility may also may indicated, along with substrate rate, time, and estimated concentrations of substrates.

The optimal conditions region of the display may indicate to the computer processor or the operator of the production facility what the substrate concentrations and other conditions (e.g., temperature, etc.) should be set to in order to optimize the chemical process and production of the chemical product. The optimal conditions region may compare the current substrate concentrations and other conditions to the best/optimal rates and values based on the hybrid model and estimation model predictions and estimations. The optimal conditions region also provides, in one or more embodiments, the correction and values after correction required to obtain an optimal target rate, as shown in the graph.

Those skilled in the art will appreciate that while the GUI (400) of FIG. 4 is shown with certain proportions of the areas displayed, plots, icons, and specific concentration information for substrates, this GUI is for purposes of example only, and embodiments disclosed herein are not limited to this specific configuration of the GUI or the substrates and concentrations shown.

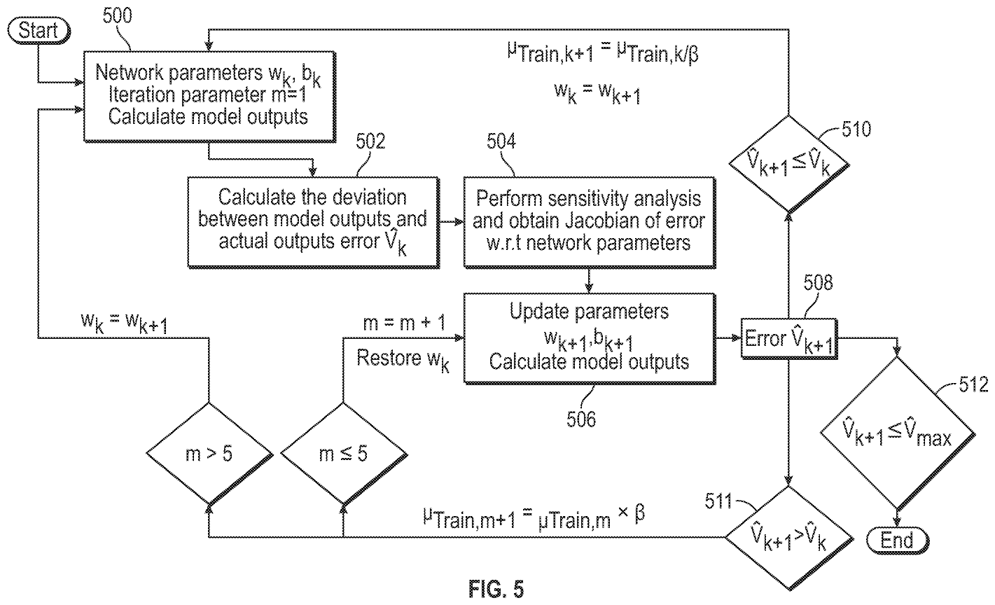

FIG. 5 shows an example flow chart of training a DNN in the hybrid model using the Levenberg-Marquardt algorithm. It is an iterative procedure wherein the parameters of the DNN are optimized. In the first step (Block 500), the parameters of the DNN are defined, and the corresponding hybrid model outputs are calculated. In the second step (Block 502), the error between the predicted model outputs and the actual outputs is calculated. In the third step (Block 504), the Jacobian matrix is calculated by deriving the sensitivity of the error value with respect to each parameter of the DNN. In the fourth step (Block 506), the DNN parameters are updated and the model outputs are predicted again. The error value (Block 508) is computed using the predicted model outputs, and is compared against the previous error value. If the error value is equal or less than the previous error value, then the combination coefficient (i.e., μ_{Train}) is decreased by a factor of β (Block 510). If the error value is greater than the previous error value, then the combination coefficient (i.e., μ_{Train}) is increased by a factor of β (Block 511). This iterative procedure as shown in FIG. 5 is repeated until the error value reaches the desired minimum value (Block 512).

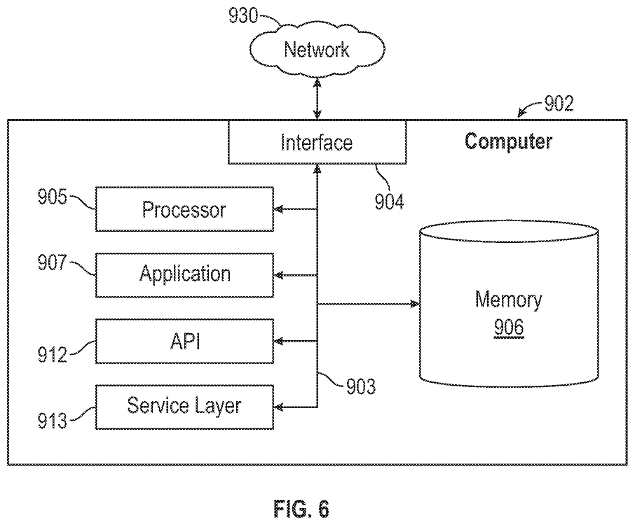

Embodiments disclosed herein may be implemented on any suitable computing device. Specifically, the information processing device (210) may be any suitable computing device capable of processing data and executing the hybrid model for simulation of a chemical process. FIG. 6 shows an example computing device in that may be implemented as information processing device 210. Specifically, FIG. 6 is a block diagram of a computer system (602) used to provide computational functionalities associated with described algorithms, methods, functions, processes, flows, and procedures as described in the instant disclosure, according to an implementation.

The illustrated computer (602) is intended to encompass any computing device such as a high performance computing (HPC) device, a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer (602) may include a computer that includes an input device, such as a keypad, keyboard, touch screen, or other device that can accept user information, and an output device that conveys information associated with the operation of the computer (602), including digital data, visual, or audio information (or a combination of information), or a GUI.

The computer (602) can serve in a role as a client, network component, a server, a database or other persistency, or any other component (or a combination of roles) of a computer system for performing the subject matter described in the instant disclosure. The illustrated computer (602) is communicably coupled with a network (630). In some implementations, one or more components of the computer (602) may be configured to operate within environments, including cloud-computing-based, local, global, or other environment (or a combination of environments).

At a high level, the computer (602) is an electronic computing device operable to receive, transmit, process, store, or manage data and information associated with the described subject matter. According to some implementations, the computer (602) may also include or be communicably coupled with an application server, e-mail server, web server, caching server, streaming data server, business intelligence (BI) server, or other server (or a combination of servers).

The computer (602) can receive requests over network (630) from a client application (for example, executing on another computer (602)) and responding to the received requests by processing the said requests in an appropriate software application. In addition, requests may also be sent to the computer (602) from internal users (for example, from a command console or by other appropriate access method), external or third-parties, other automated applications, as well as any other appropriate entities, individuals, systems, or computers.

Each of the components of the computer (602) can communicate using a system bus (603). In some implementations, any or all of the components of the computer (602), both hardware or software (or a combination of hardware and software), may interface with each other or the interface (604) (or a combination of both) over the system bus (603) using an application programming interface (API) (612) or a service layer (613) (or a combination of the API (612) and service layer (613). The API (612) may include specifications for routines, data structures, and object classes. The API (612) may be either computer-language independent or dependent and refer to a complete interface, a single function, or even a set of APIs. The service layer (613) provides software services to the computer (602) or other components (whether or not illustrated) that are communicably coupled to the computer (602). The functionality of the computer (602) may be accessible for all service consumers using this service layer. Software services, such as those provided by the service layer (613), provide reusable, defined business functionalities through a defined interface. For example, the interface may be software written in JAVA, C++, or other suitable language providing data in extensible markup language (XML) format or other suitable format. While illustrated as an integrated component of the computer (602), alternative implementations may illustrate the API (612) or the service layer (613) as stand-alone components in relation to other components of the computer (602) or other components (whether or not illustrated) that are communicably coupled to the computer (602). Moreover, any or all parts of the API (612) or the service layer (613) may be implemented as child or sub-modules of another software module, enterprise application, or hardware module without departing from the scope of this disclosure.

The computer (602) includes an interface (604). Although illustrated as a single interface (604) in FIG. 6, two or more interfaces (604) may be used according to particular needs, desires, or particular implementations of the computer (602). The interface (604) is used by the computer (602) for communicating with other systems in a distributed environment that are connected to the network (630). Generally, the interface (604 includes logic encoded in software or hardware (or a combination of software and hardware) and operable to communicate with the network (630). More specifically, the interface (604) may include software supporting one or more communication protocols associated with communications such that the network (630) or interface's hardware is operable to communicate physical signals within and outside of the illustrated computer (602).

The computer (602) includes at least one computer processor (605). Although illustrated as a single computer processor (605) in FIG. 6, two or more processors may be used according to particular needs, desires, or particular implementations of the computer (602). Generally, the computer processor (605) executes instructions and manipulates data to perform the operations of the computer (602) and any algorithms, methods, functions, processes, flows, and procedures as described in the instant disclosure.

The computer (602) also includes a memory (606) that holds data for the computer (602) or other components (or a combination of both) that can be connected to the network (630). For example, memory (606) can be a database storing data consistent with this disclosure. Although illustrated as a single memory (606) in FIG. 6, two or more memories may be used according to particular needs, desires, or particular implementations of the computer (602) and the described functionality. While memory (606) is illustrated as an integral component of the computer (602), in alternative implementations, memory (606) can be external to the computer (602).

The application (607) is an algorithmic software engine providing functionality according to particular needs, desires, or particular implementations of the computer (602), particularly with respect to functionality described in this disclosure. For example, application (607) can serve as one or more components, modules, applications, etc. Further, although illustrated as a single application (607), the application (607) may be implemented as multiple applications (607) on the computer (602). In addition, although illustrated as integral to the computer (602), in alternative implementations, the application (607) can be external to the computer (602).

There may be any number of computers (602) associated with, or external to, a computer system containing computer (602), each computer (602) communicating over network (630). Further, the term “client,” “user,” and other appropriate terminology may be used interchangeably as appropriate without departing from the scope of this disclosure. Moreover, this disclosure contemplates that many users may use one computer (602), or that one user may use multiple computers (602).

In some embodiments, the computer (602) is implemented as part of a cloud computing system. For example, a cloud computing system may include one or more remote servers along with various other cloud components, such as cloud storage units and edge servers. In particular, a cloud computing system may perform one or more computing operations without direct active management by a user device or local computer system. As such, a cloud computing system may have different functions distributed over multiple locations from a central server, which may be performed using one or more Internet connections. More specifically, cloud computing system may operate according to one or more service models, such as infrastructure as a service (IaaS), platform as a service (PaaS), software as a service (SaaS), mobile “backend” as a service (MBaaS), serverless computing, artificial intelligence (AI) as a service (AIaaS), and/or function as a service (FaaS).

The term “major” can mean an amount of about 50%, such as about 80%, by weight of a compound or class of compounds in a stream.

The term “substantially” can mean an amount of about 80%, such as about 90%, or about 99%, by mole, of a compound or class of compounds in a stream.

As used here and in the appended claims, the words “comprise,” “has,” and “include” and grammatical variations thereof are each intended to have an open, non-limiting meaning that does not exclude additional elements or steps.

“Optionally” means that the subsequently described event or circumstances may or may not occur. The description includes instances where the event or circumstance occurs and instances where it does not occur.

When the word “approximately” or “about” are used, this term may mean that there can be a variance in value of up to ±10%, of up to 5%, of up to 2%, of up to 1%, of up to 0.5%, of up to 0.1%, or up to 0.01%.

Ranges may be expressed as from about one particular value to about another particular value, inclusive. When such a range is expressed, it should be understood that another one or more embodiments is from the one particular value to the other particular value, along with all particular values and combinations thereof within the range.

Although a few example embodiments have been described in detail, those skilled in the art will readily appreciate that many modifications are possible in the example embodiments without materially departing from this disclosure. All modifications of one or more disclosed embodiments are intended to be included within the scope of this disclosure as defined in the following claims. In the claims, means-plus-function clauses are intended to cover the structures previously described as performing the recited function and not only structural equivalents, but also equivalent structures. It is the express intention of the applicant not to invoke 35 U.S.C. § 112(f) for any limitations of any of the claims, except for those in which the claim expressly uses the words ‘means for’ together with an associated function.

It is noted that one or more of the following claims utilize the term “where” or “in which” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.” For the purposes of defining the present technology, the transitional phrase “consisting of” may be introduced in the claims as a closed preamble term limiting the scope of the claims to the recited components or steps and any naturally occurring impurities. For the purposes of defining the present technology, the transitional phrase “consisting essentially of” may be introduced in the claims to limit the scope of one or more claims to the recited elements, components, materials, or method steps as well as any non-recited elements, components, materials, or method steps that do not materially affect the novel characteristics of the claimed subject matter. The transitional phrases “consisting of” and “consisting essentially of” may be interpreted to be subsets of the open-ended transitional phrases, such as “comprising” and “including,” such that any use of an open-ended phrase to introduce a recitation of a series of elements, components, materials, or steps should be interpreted to also disclose recitation of the series of elements, components, materials, or steps using the closed terms “consisting of” and “consisting essentially of.” For example, the recitation of a composition “comprising” components A, B, and C should be interpreted as also disclosing a composition “consisting of” components A, B, and C as well as a composition “consisting essentially of” components A, B, and C. Any quantitative value expressed in the present application may be considered to include open-ended embodiments consistent with the transitional phrases “comprising” or “including” as well as closed or partially closed embodiments consistent with the transitional phrases “consisting of” and “consisting essentially of.” The words “comprise,” “has,” and “include” and grammatical variations thereof are each intended to have an open, non-limiting meaning that does not exclude additional elements or steps.

While one or more embodiments of the present disclosure have been described with respect to a limited number of embodiments, those skilled in the art, having benefit of this disclosure, will appreciate that other embodiments can be devised, which do not depart from the scope of the disclosure. Accordingly, the scope of the disclosure should be limited only by the attached claims.

What is claimed is:

1. A system for producing a product, the system comprising:
a production facility for producing a product using a chemical process involving chemical reactions; and
an information processing device comprising a computer processor that:
simulates, using a hybrid model, the chemical reactions in the chemical process that produces the product in order to obtain a predicted output, wherein the hybrid model is a combination of a first-principles model and a data-driven model,
determines, using an observer model, expected concentrations and levels of all substrates for the simulated process,
sets derived optimal conditions for the chemical process based on the estimated concentrations and levels of all substrates, and
predicts future production results based on a current status of the production facility.

2. The system of claim 1, wherein the chemical process is fermentation and wherein the product is a micro-organism or its metabolites.

3. The system of claim 1, wherein the data-driven model in the hybrid model comprises a deep neural network (DNN) model.

4. The system of claim 1, wherein the observer model is configured to estimate the current status of the production facility.

5. The system of claim 1, wherein the observer model is configured to estimate the current status of the production facility, and employs an open-loop multi-rate observer based on the hybrid model, where states are re-initialized when measurements are available.

6. The system of claim 1, wherein the information processing device is further configured to generate one or more production condition candidates each of which optimizes the production facility by maximizing the productivity and/or minimizing the operating cost.

7. The system of claim 1, wherein the current status represents the quality of the current batch process run at the production facility.

8. The system of claim 2, wherein the derived optimal conditions for the chemical process comprise adjusted feed rates of a substrate, a catalyst fed into the reactor, and a temperature of the reactor.

9. The system of claim 1, further comprising: a GUI dashboard for displaying the current status of the production facility, the estimation of substrate concentrations, and the optimal conditions for the chemical process.

10. A computer-implemented method for predicting substrate concentrations in a chemical process to produce a chemical product, comprising:
simulating, using a hybrid model, chemical reactions in the chemical process to obtain a predicted output, wherein the hybrid model is a combination of a first-principles model and a data-driven model;
determining, using an observer model, an estimation of all concentrations and levels for the simulated chemical process;
setting derived optimal conditions for the chemical process based on the estimated substrate concentrations, and
predicting future production results of the product based on a current status of the production facility.

11. The computer-implemented method of claim 10, further comprising: displaying the current status of the production facility, the estimated substrate concentrations and the optimal conditions for the chemical process on a GUI dashboard, wherein the derived optimal conditions are set based on information displayed on the GUI dashboard.

12. The computer-implemented method of claim 10, wherein the current status represents the quality of the current batch process run at the production facility.

13. The computer-implemented method of claim 10, wherein the derived optimal conditions for the chemical process comprise adjusted feed rates of a substrate, a catalyst fed into a reactor, and temperature of a reactor in which the chemical process is performed.

14. The computer-implemented method of claim 10, wherein the chemical process is fermentation and wherein the product is micro-organism or its metabolites.

15. The computer-implemented method of claim 10, wherein the data-driven model in the hybrid model comprises a deep neural network (DNN) model.
